(19) United States
(12) Patent Application Publication
(10) Pub. No.: US 2005/0266393 A1
(43) Pub. Date: Dec. 1, 2005

(54) Circulating flow device for assays of cell cultures, cellular components and cell products

(76) Inventors: Gregory T. Baxter, Palo Alto, CA (US); Robert Freedman, Beverly Hills, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/954,720

(22) Filed: Sep. 30, 2004

Related U.S. Application Data
(60) Provisional application No. 60/507,877, filed on Oct. 1, 2003.

(51) Int. Cl. C12Q 1/00; C12N 5/02

(52) U.S. Cl. 435/4; 435/404; 435/289.1

(57) ABSTRACT

In vitro culture devices and methods are described. The subject methods and devices provide a means whereby cells and/or subcellular material are grown or held in a culture device that maintains the cells and/or subcellular material in a physiologically representative environment, thereby improving the predictive value of toxicity and metabolism assays, and the relevance of experimental results derived from such assays to actual in vivo conditions, processes and outcomes. The culture devices of the invention comprise a fluidic channel connected to or otherwise integrated with at least one chamber, preferably integrated in a chip format. The specific chamber geometry is designed to provide cellular interactions, liquid flow, and liquid residence and other parameter values that correlate with those found in or produced by the corresponding cell, organs or tissues, or components or products thereof, in vivo. Each device comprises at least one chamber and at least one inlet and one outlet port that allow for recirculation of the culture medium. The device will usually include a mechanism for obtaining signals from the cells and culture medium.

### CLAIM FOR PRIORITY

This application claims priority under 35 U.S.C. § 119(e) of U.S. Provisional Application No. 60/507,877, filed Oct. 1, 2003, titled “CIRCULATING FLOW DEVICE FOR ASSAYS OF CELL CULTURES, CELLULAR COMPONENTS AND CELL PRODUCTS” which is incorporated herein by reference in its entirety.

### BACKGROUND OF THE INVENTION

1. Field of the Invention

The invention relates to in vitro culturing systems.

2. Description of the Related Art

Pharmacokinetics is the study of the fate of pharmaceuticals and other biologically active compounds from the time they are introduced into the body until they are eliminated. For example, the sequence of events for an oral drug can include absorption through the various mucosal surfaces, distribution via the blood stream to various tissues, biotransformation in the liver and other tissues, action at the target site, and elimination of drug or metabolites in urine or bile. Pharmacokinetics provides a rational means of approaching the metabolism of a compound in a biological system. For reviews of pharmacokinetic equations and models, see, for example, Poulin and Theil (2000) J Pharm Sci. 89(1):16-35; Slob et al. (1997) Crit Rev Toxicol. 27(3):261-72; Haddad et al. (1996) Toxicol Lett. 85(2):113-26; Hoang (1995) Toxicol Lett. 79(1-3):99-106; Knaak et al. (1995) Toxicol Lett. 79(1-3):87-98; and Ball and Schwartz (1994) Comput Biol Med. 24(4):269-76.

One of the fundamental challenges researchers face in drug, environmental, nutritional, consumer product safety, and toxicology studies is the extrapolation of metabolic data and risk assessment from in vitro cell culture assays to animals. Although some conclusions can be drawn with the application of appropriate pharmacokinetic principles, there are still substantial limitations. One concern is that current screening assays utilize cells under conditions that do not replicate their function in their natural setting. The circulatory flow, interaction with other tissues, and other parameters associated with a physiological response are not found in standard tissue culture formats. Therefore, the resulting assay data is not based on the pattern of drug or toxin exposure that would be found in an animal.

Within living beings, concentration, time, and metabolism interact to influence the intensity and duration of a pharmacologic or toxic response. For example, in vivo the presence of liver function strongly affects drug metabolism and bioavailability. Elimination of an active drug by the liver occurs by biotransformation, and excretion. Biotransformation reactions include reactions catalyzed by the cytochrome P450 enzymes, which transform many chemically diverse drugs. A second biotransformation phase can add a hydrophilic group, such as glutathione, glucuronic acid or sulfate, to increase water solubility and speed elimination through the kidneys.

While biotransformation can be beneficial, it may also have undesirable consequences. Toxicity results from a complex interaction between a compound and the organism. During the process of biotransformation, the resulting metabolite can be more toxic than the parent compound. The biotransformation process of a compound in an organism is dynamic, each metabolic product has a specific half-life dependent on the circulatory residence time within the liver and the circulatory transit time within the body. The static, single-cell assays traditionally used for toxicity screening fail to replicate the physiological nature of the liver organ within the body of a living organism.

U.S. Pat. No. 5,612,188 issued to Shuler et al. describes a multicompartmental cell culture system. This culture system uses large components, such as culture chambers, sensors, and pumps, which require the use of large quantities of culture media, cells and test compounds. This system is very expensive to operate and requires a large amount of space in which to operate. Because this system is on such a large scale, the physiological parameters vary considerably from that found in an in vivo situation.

The development of microscale screening assays and devices that can provide better, faster and more efficient prediction of in vivo toxicity, metabolism, and clinical drug performance is of great interest in a number of fields, and is addressed in the present invention.

### SUMMARY OF THE INVENTION

An in vitro culture device is described. The device permits cells, subcellular material, cell products, or subcellular components to be maintained in vitro. In one embodiment, the culture device maintains these elements under conditions characterized by physiological parameter values comparable to or simulative of those found in vivo and determined through the application of a mathematical model of physiological process(es). In another embodiment, the culture device maintains these elements under conditions with pharmacokinetic parameter values similar to those found in vivo determined through the application of a specific physiologically-based pharmacokinetic (“PBPK”) model. Pharmacokinetic parameters of interest include interactions between cells and/or their subcellular material, subcellular components or cellular products, liquid residence time, liquid to cell ratios, metabolism by cells, shear stress, circulatory flow distribution, circulatory transit time, and the like.

By providing a physiologically-based culture system that mimics the natural state of cells within a specific organ or tissue and within a living organism, the predictive value of screening and toxicity assays—e.g., the accuracy with which such in vitro tests can predict pharmacokinetics, pharmacodynamics, efficacy, absorption, distribution, metabolism, excretion, toxicity, bioavailability, biotransformation, and other physiological or pharmacokinetic conditions, processes and outcomes as found in vivo—is enhanced.

In another embodiment of the present invention, the culture device maintains the cells, or subcellular material such as cellular products or subcellular components, under conditions where the values of one or more pharmacokinetic parameters mimic or simulate the value of that parameter, or, as the case may be, the values of those parameters, as found in vivo. In yet another embodiment, the culture device maintains the cells or subcelluar material under conditions where the values obtained for one or more pharmacokinetic parameters deviate from those values found in vivo. For example, the liquid residence time may be deliberately reduced in order to obtain more rapid results.

In an embodiment of the present invention, the geometry of the culture device comprises the physical dimensions of the chamber, chambers, channel, channels, and any other component parts of the device, the internal topographical features of component parts of the device such as flat surfaces, pillars, ridges, microcarrier beads and the like, the relative arrangement, interconnection or integration one to another of the component parts of the device, and also the flow rate of fluid in and through the device. By virtue of its causing the simulation of at least one physiological parameter with a value comparable to a value obtained for that parameter in vivo, the geometry of the device tangibly embodies specific physiological information.

In one embodiment, the present invention comprises a channel or channels connecting to or otherwise integrated with at least one chamber. The specific chamber geometry is designed to provide cellular interactions, liquid flow rate, and liquid residence parameters that correlate with those found in vivo for the corresponding cells, tissue, or organ that particular chamber simulates. The fluidics and channels are designed to accurately represent primary elements of the circulatory or lymphatic systems. These components may be integrated into a chip format. The design and validation of these geometries is based on a physiologically-based pharmacokinetic (“PBPK”) model, e.g., a mathematical model that represents the body, or body systems or components, as interconnected compartments representing different organs or tissues. In another embodiment, the design and validation of these geometries is based on a mathematical model other than a PBPK model. In other embodiments, the design and validation of the device geometry can be based on mathematical models other than a PBPK model such as a pharmacokinetic/pharmacodynamic (“PK/PD”) model, a drug clearance model, or other form of mathematical model. Drug clearance models are mathematical models used to predict the length of time a drug remains in the body and/or the rate of elimination of a drug from the blood. A PK/PD model is a mathematical model used to predict the action of a drug in a living system based on pharmacokinetic information derived from in silico, in vitro or animal data.

In one embodiment of the present invention, the chamber of the device can be seeded with the appropriate cells. For example, a chamber designed to provide liver pharmacokinetic parameters is seeded with hepatocytes. The result is a pharmacokinetic-based cell culture system that accurately represents, for example, tissue-to-blood volume ratio and drug residence time in the liver of the animal species it is modeling. Such a device would be applicable for the rapid and accurate determination of drug metabolism. In an alternative embodiment, the chamber can contain subcellular material. Subcellular material can be subcellular components, such as mitochondria, microsomes and the like. For example, a chamber designed to provide liver enzyme metabolizing activity might contain isolated liver microsomes. Alternatively, subcellular material can be cellular products, such as enzymes, nucleic acids, and the like. For example, a chamber designed to provide liver cytochrome P450 enzyme activity might contain immobilized liver cytochrome P450 enzyme(s). In one embodiment, the chamber can contain cellular material. Cellular material can be either cells or subcellular material and can be either naturally occurring or man-made.

The cellular products can be derived from an appropriate mammalian cell or they can be synthetic. An example of a synthetic cellular product would be an enzyme which differs in structure and/or activity from the naturally occurring enzyme through a process of genetic manipulation or chemical synthesis. The subcellular components can be derived from an appropriate mammalian cell or they can be synthetic. An example of a synthetic subcellular component would be an artificial microsome.

In an alternative embodiment, the chamber can contain a combination of cultured cells, subcellular components, and cellular products. In yet another embodiment, the chamber may contain a confluent monolayer of gastrointestinal epithelial cells positioned in the device such that fluid may flow along either side of but not through the monolayer, and the intervening cell layer thus provides a barrier to fluid flow. Such a device would be applicable in determining absorption characteristics of an orally administered drug. In various, other embodiments of the present invention, the cells, cellular components, cellular products, or various combinations thereof as the case may be, may be adherent to the chamber or alternatively they may be free to circulate within the device; or alternatively, some may be adherent while others circulate.

The present invention provides a culture device comprising a chamber containing cultured cells or subcellular materials (e.g., subcellular components or cellular products), wherein the chamber also comprises an inlet and an outlet 105 for flow of culture medium. The culture device may contain channels connecting to or otherwise interfacing with the chamber or the inlet and/or outlet. The culture device may contain circulating or adherent cells, wherein the cells may be eukaryotic (e.g., plant or animal; mammalian, primary, tumor or genetically altered cells), prokaryotic, or viral. In one embodiment, the culture device is microscale, meaning one or more feature(s) of the device measure one millimeter or less in one or more dimension(s) (e.g., length, width, or depth). In another embodiment, the device may be larger than microscale.

In one embodiment, the geometry and design of the present invention are contrived so as to provide that the value obtained for at least one physiological parameter is comparable to the value obtained for that parameter in vivo. For example, at least one of the physiological parameters of the present invention may be the liquid residence time, liquid-to-cell volume ratio, circulatory transit time, circulatory flow distribution, metabolism by cells, shear stress, or the like. In another embodiment of the present invention, the geometry and design of the culture device are contrived so as to produce values for one or more physiological parameters, none of which are intended to be comparable to values produced in vivo.

An embodiment of the present invention may contain a single compartment (e.g., a chamber); or alternatively, another embodiment of the present invention may contain two compartments (e.g., chambers), where one compartment contains cells, subcellular components, or cellular products and the other compartment is an open reservoir for the addition or withdrawal of culture media. In another embodiment of the present invention, the culture device may contain three compartments, where one compartment contains cells, subcellular components, or cellular products, one compartment is an open reservoir for the addition or withdrawal of culture media, and one compartment contains a pumping mechanism. The culture device may further comprise culture medium wherein the culture medium may flow through the chamber(s) and device once, or alternatively, the culture medium may re-circulate through the chamber(s) and device. Another embodiment of the present invention may further comprise a pumping mechanism, wherein the pumping mechanism may either be integrated in the device or separate from the device. In one such embodiment, the pumping mechanism may be electrokinetic or, alternatively, an alternative embodiment may comprise a diaphragm pump that is mechanically actuated or pneumatically actuated. In another embodiment, the culture device may further comprise a debubbler located within the device or external to the device. In another embodiment of the present invention, the culture device may comprise at least one sensor for obtaining signals from the cultured cells, subcellular components, or cellular products, wherein at least one sensor may be a biosensor and the biosensor may comprise a waveguide.

In one embodiment, the culture device may be microfabricated, or manufactured from a microfabricated master, such as a silicon master. In one embodiment, the method of microfabrication may comprise mass production of devices made of silicon, by techniques such as plasma-etch and the like. In one embodiment, the method of microfabrication may comprise mass production of devices made of polymeric material, by techniques such as embossing, injection molding, and the like. In one embodiment, the chamber may provide for three-dimensional growth of cells. In one embodiment, the chamber may contain a plurality of cell types, a tissue biopsy, or a section of a tissue or organ. In one embodiment, the chamber may comprise or contain an artificial tissue construct, such as an artificial liver tissue construct, an artificial kidney tissue construct, an artificial cardiac tissue construct, an artificial blood-brain barrier construct, an artificial intestinal tissue construct, an artificial corneal tissue construct, or the like. In one embodiment, the chamber may contain one or more cellular products, wherein the cellular product(s) is one or a plurality of expressions of an enzyme, nucleic acid, protein, lipid, carbohydrate, or the like. In one embodiment, the chamber may contain one or more subcellular components, wherein the subcellular component(s) is one or a plurality of expressions of a microsome, mitochondrion, nucleus, ribosome, organelle, plasma membrane, and the like. In one embodiment, the present invention may comprise multiple interconnected devices.

An embodiment of the present invention may provide a method for determining the effect of an input variable on the culture device, wherein the method may in part comprise contacting the culture device with an input variable and monitoring at least one output parameter. In one embodiment of the present invention, the method of monitoring at least one output parameter may comprise obtaining information from at least one sensor in the device, wherein the input variable may be an organic compound, an inorganic compound, a complex sample, a pharmaceutical sample, an environmental sample, a nutritional sample, a consumer product, an industrial chemical, a biologically derived compound, or a biological or chemical warfare agent.

In another embodiment of the present invention, the culture device may be a configuration wherein the chamber and the connecting channels are one and the same.

In one embodiment, a culture device comprising at least one microscale chamber is configured to hold subcellular material, wherein the microscale chamber comprises an inlet and an outlet for fluid flow and wherein the microscale chamber is configured to simulate in vitro one or more physiological parameters derived from a mathematical model. In the following embodiments a variety of alternatives are also disclosed.

For example, the culture device, by virtue of its causing the simulation of at least one physiological parameter with a value comparable to a value obtained for that parameter in vivo, the geometry of the device tangibly embodies specific physiological information. The mathematical model used in the culture device may be a physiologically-based pharmacokinetic model, or a single-compartment pharmacokinetic model, or a multi-compartment pharmacokinetic model, or a non-linear pharmacokinetic model, or a drug clearance model, or the like. The physiological parameter may be a pharmacokinetic parameter. The geometry of the chamber may cause the culture device to simulate at least one pharmacokinetic parameter with a value comparable to a value obtained in vivo. The flow rate of fluid through the chamber may simulate at least one physiological parameter with a value comparable to a value obtained in vivo.

The culture device may further comprise a second microscale chamber in fluidic communication with the first microscale chamber, wherein the second microscale chamber comprises an open reservoir for the addition or withdrawal of culture medium. The culture device may further comprise a third microscale chamber in fluidic communication with the first and second microscale chambers, wherein the third microscale chamber comprises a pumping mechanism.

The culture device may further comprise a culture medium. The culture medium within the culture device may flow through the microscale chamber. The culture medium may re-circulate through the microscale chamber.

The culture device may further comprise a pumping mechanism. The pumping mechanism may be integrated in the culture device. The pumping mechanism may be electrokinetic. The pumping mechanism may be a diaphragm pump. The pumping mechanism may be mechanically actuated. The pumping mechanism may be pneumatically actuated. The pumping mechanism may be external to the device.

The culture device may further comprise a microfluidic channel in communication with the microscale chamber. The microscale chamber and the microfluidic channel may be one and the same. The microfluidic channel may comprise a debubbler located therein. The culture device may comprise a debubbler that is located externally to the device.

The culture device may include at least one pharmacokinetic parameter selected from the group consisting of liquid residence time, liquid to cell volume ratio, organ/tissue size ratio, circulatory transit time, circulatory flow distribution, and metabolism by cells. The culture device may further comprise at least one sensor for obtaining signals from the cellular medium. The sensor may be a biosensor. The sensor may comprise a waveguide.

The culture device may be microfabricated. The culture device may be manufactured from a microfabricated master. The culture device may be manufactured by mass production that causes the geometry of the device (including the provision for the rate of fluid flow in and through the device), and therefore the information embodied in the device, to be substantially the same from one such manufactured copy, specimen or iteration of the device to the next. The process of mass production may include that the device is manufactured from a microfabricated master.

The chamber of the culture device may provide for three-dimensional growth of cells. The microscale chamber may contain a plurality of cell types. The microscale chamber may contain a tissue biopsy. The microscale chamber may contain a cross-section of a tissue or organ. The microscale chamber may contain an artificial tissue construct.

The subcellular material in the culture device may be a cellular product. The cellular product may be selected from the group consisting of an enzyme, a nucleic acid, a protein, a lipid, and a carbohydrate. The cellular product may be man-made. The cellular product comprises a naturally occurring or man-made cellular product in conjunction with some other biochemical entity. The subcellular material may comprise a subcellular component. The subcellular component may be a microsome, mitochondrion, nucleus, ribosome, plasma membrane, and the like. The subcellular component may be man-made. The subcellular component may comprise a naturally occurring or man-made subcellular component in conjunction with some other biochemical entity. The culture device may comprise multiple interconnected culture devices.

In one embodiment, a method for culturing subcellular material comprises receiving subcellular material within a microscale chamber, wherein the microscale chamber comprises an inlet and an outlet for fluid flow, and wherein the fluid flows through the microscale chamber; and simulating in vitro one or more physiological parameters derived from a mathematical model. The mathematical model of the method may be a physiologically-based pharmacokinetic model. The physiological parameter may be a pharmacokinetic parameter.

The act of simulating may simulate at least one pharmacokinetic parameter with a value comparable to a value obtained in vivo. The method may supply the culture medium within the microscale chamber from a second microscale chamber in fluidic communication with the first microscale chamber, wherein the second microscale chamber comprises an open reservoir. The method may re-circulate a culture medium through the microscale chamber. At least one pharmacokinetic parameter may be selected from the group consisting of liquid residence time, liquid to cell ratio, circulatory transit time, or metabolism by cells.

The method may further comprise contacting the culture system with an input variable; and monitoring at least one output parameter. The act of monitoring the output parameter may comprise obtaining information from at least one sensor. The input variable may be an organic compound. The input variable may be an inorganic compound. The input variable is a complex sample. The input variable may be selected from the group consisting of a pharmaceutical, environmental sample, a nutritional sample, or a consumer product, industrial chemical, biologically derived compound, biological and chemical warfare agent. In addition, the method may comprise sensing the condition of the cellular medium.

In another embodiment, a culture device comprises at least one microscale chamber that is configured to hold cellular material, wherein the microscale chamber comprises an inlet and an outlet for fluid flow and wherein the microscale chamber is configured to simulate in vitro one or more physiological parameters derived from a mathematical model; a first sensor located upstream of the inlet of the microscale chamber; a second sensor located downstream of the outlet of the microscale chamber; and a culture medium that flows through the inlet and outlet of the microscale chamber.

The first and second sensors may be integrated buried waveguides. At least one of the first and second sensors may be a biosensor. The biosensor may provide information on cellular metabolism. The biosensor may provide information on enzyme activity. The first and second sensors may be configured to monitor the culture medium. The first and second sensors may be configured to monitor one of the group consisting of oxygen, carbon dioxide, and pH of the culture medium. The first and second sensors may be configured to control gas levels within the microscale chamber.

In one embodiment, a method for culturing cellular material comprises receiving cellular material in at least one microscale chamber, wherein the microscale chamber comprises an inlet and an outlet for fluid flow; simulating in vitro one or more physiological parameters derived from a mathematical model; sensing culture medium with a first sensor located upstream of the inlet of the microscale chamber; and sensing the culture medium with a second sensor located downstream of the outlet of the microscale chamber.

At least one of the acts of sensing may obtain information on cellular metabolism. At least one of the acts of sensing may obtain information on enzyme activity. At least one of the acts of sensing may monitor the culture medium. At least one of the acts of sensing may monitor one of the group consisting of oxygen, carbon dioxide, and pH of the culture medium. At least one of the acts of sensing may control gas levels within the microscale chamber.

In another embodiment, a culture device comprises at least one microscale chamber that is configured to hold cellular material, wherein the microscale chamber comprises an inlet and an outlet for fluid flow and wherein the microscale chamber is configured to simulate in vitro one or more physiological parameters derived from a mathematical model; a fluid channel in fluidic communication with either the inlet or outlet of the microscale chamber; and one or more electrodes in communication with the fluid channel, the one or more electrodes configured to induce fluid flow within the fluid channel.

The culture device may further comprise a voltage source that is configured to alternate the sequence of voltage applied to the electrodes to induce directional flow of the fluid within the fluid channel. The electrodes may induce eletrokinetic flow. The electrodes may induce eletroosmotic flow.

In another embodiment, a method for culturing cellular material comprises holding cellular material in at least one microscale chamber, wherein the microscale chamber comprises an inlet and an outlet for fluid flow; simulating in vitro one or more physiological parameters derived from a mathematical model; and altering voltage in one or more electrodes to induce flow fluid through the microscale chamber.

The act of alternating may alternate the sequence of voltage applied to the electrodes to induce directional flow of the fluid within a fluid channel that is in fluidic communication with the microscale chamber. The act of altering voltage may induce eletrokinetic flow. The act of altering voltages may induce eletroosmotic flow.

In one embodiment, a culture device comprises at least one microscale chamber that is configured to hold cellular material, wherein the microscale chamber comprises an inlet and an outlet for fluid flow and wherein the microscale chamber is configured to simulate in vitro one or more physiological parameters derived from a mathematical model; and at least one reservoir in fluidic communication with the microscale chamber, the reservoir comprising a flexible membrane, wherein depressing the flexible membrane induces fluid flow into the microscale chamber.

The flexible membrane may comprise silicon at least in part. The flexible membrane may recirculate fluid flow between the microscale chamber and the reservoir. The flexible membrane may recirculate fluid flow between the microscale chamber and the reservoir. Multiple reservoirs may be in fluidic communication and at least one of the multiple reservoirs may comprise the flexible membrane.

In another embodiment a method for culturing cellular material comprises holding cellular material within at least one microscale chamber wherein the microscale chamber comprises an inlet and an outlet for fluid flow; simulating in vitro one or more physiological parameters derived from a mathematical model; and inducing fluidic flow within the microscale chamber by depressing a flexible membrane.

The flexible membrane may be attached to a reservoir that is in fluidic communication with the microscale chamber. The flexible membrane may comprise silicon at least in part. The act of inducing fluidic flow may recirculate fluid flow between the microscale chamber and a reservoir.

In one embodiment, a culture device comprises at least one microscale chamber that is configured to hold cellular material, wherein the microscale chamber comprises an inlet and an outlet for fluid flow and wherein the microscale chamber is configured to simulate in vitro one or more physiological parameters derived from a mathematical model; and a culture medium within the microscale chamber, the culture medium comprising microscale magnetic particles.

The culture device may further comprise a rotating magnetic field that induces a circular flow of the culture medium within the microscale chamber. The culture device may further comprise a magnetic field that induces a flow of the culture medium within the microscale chamber. The culture device may further comprise a gas permeable membrane that encloses at least a portion of the microscale chamber.

In another embodiment, a method for culturing cellular material comprises holding cellular material in at least one microscale chamber, wherein the microscale chamber comprises an inlet and an outlet for fluid flow; simulating in vitro one or more physiological parameters derived from a mathematical model; and adding a culture medium to the microscale chamber wherein the culture medium comprises microscale magnetic particles.

The method may further comprise rotating a magnetic field to induce a circular flow of the culture medium within the microscale chamber. The method may further comprise inducing a magnetic field that induces a flow of the culture medium within the microscale chamber. The method may comprise enclosing at least a portion of the microscale chamber with a gas permeable membrane.

### BRIEF DESCRIPTION OF THE DRAWINGS

FIG. 1 is a schematic view of one embodiment of the exterior of the system of the present invention.

FIG. 2 is a schematic view of another embodiment of the system of the present invention.

FIG. 3 is a schematic view of yet another embodiment of the system of the present invention.

FIG. 4 is a schematic view of yet another embodiment of the system of the present invention.

FIG. 5 is a schematic view of yet another embodiment of the system of the present invention.

FIG. 6 is a schematic view of yet another embodiment of the system of the present invention.

FIG. 7 is a schematic view of yet another embodiment of the system of the present invention.

FIG. 8 is a schematic view of yet another embodiment of the system of the present invention.

### DETAILED DESCRIPTION OF THE PREFERRED EMBODIMENT

In one embodiment of the present invention, the in vitro culture device provides a means whereby cells, subcellular material, subcellular components, or cell products are maintained in vitro in an environment physiologically representative of certain in vivo conditions, thereby improving the accuracy with which toxicity and metabolic assays performed on the device are able to predict physiological outcomes obtained in vivo. In one embodiment, a pharmacokinetic culture device is seeded with the appropriate cells, thereby creating a culture system which can then be used for compound toxicity assays, metabolism studies, absorption studies, bioavailability studies, models for development of cells of interest, models of infection kinetics, immunology studies, and the like. An input variable, which may be, for example, a compound, sample, genetic sequence, pathogen, cell, (such as a progenitor cell) is added to an established culture system. Various cellular outputs may be assessed to determine the response of the cells to the input variable, including pH of the medium, concentration of O₂ and CO₂ in the medium, expression of proteins and other cellular markers, cell viability, or release of cellular products into the culture medium.

In the following detailed description of the preferred embodiments, reference is made to the accompanying drawings that form a part hereof, and in which are shown by way of illustration specific embodiments in which the invention may be practiced. It is to be understood that other embodiments may be utilized and structural changes may be made without departing from the scope of the present invention.

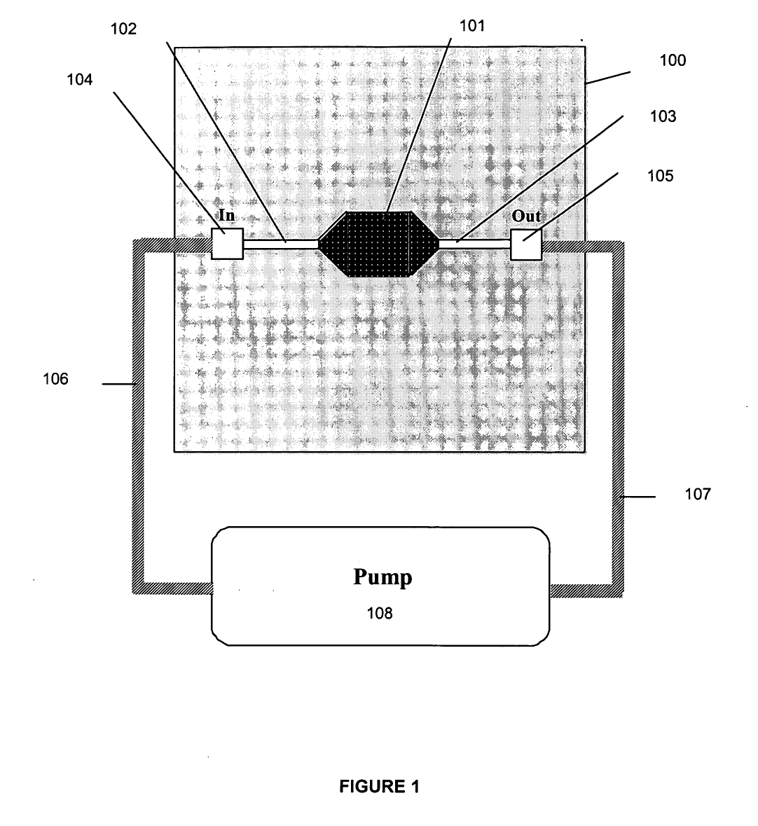

FIG. 1 is a schematic view of one embodiment of the system of the present invention. The system includes a culture chamber 101 formed on a substrate of silicon, which is commonly referred to as a chip 100. It should be noted that more than one culture chamber 101 could be housed or formed on a single chip 100. The chamber 101 has an inlet 104 and an outlet 105. The inlet 104 is located at one end of the chamber 101 and the outlet 105 is located at the other end of the chamber 101. The inlet 104 and outlet 105 are connected to the chamber 101 by a fluid path, the inlet channel 102 and the outlet channel 103, respectively. The system includes a pump 108 for circulating the fluid in the system. A microtube 107 connects between the outlet side of the pump 108 and the outlet 105 and another microtube 106 connects between the inlet side of the pump 108 and the inlet 104. In one embodiment, the chamber 101, the fluid path, and the pump 108 form the system. The system may also include additional chambers 101.

In one embodiment, the design and geometry of (including the rate and volume of fluid flow through) the device is derived from a PBPK model and thus provides for the particular conditions of cell culture, cell growth, pharmacokinetics, pharmacodynamics, and microfluidic operation that obtain in that certain embodiment of the invention. Each device comprises at least one chamber 101, an inlet 104, and an outlet 105 so that the culture medium can be circulated.

In another embodiment of the present invention, the features of design and geometry that determine the particular conditions of cell culture, cell growth, pharmacokinetics, pharmacodynamics, and microfluidic operation that obtain in the device are derived from a mathematical model that is other than a PBPK model.

In yet another embodiment of the present invention, the design and geometry of the device are contrived with the intention of creating an environment that is physiologically representative of no particular in vivo conditions.

In one embodiment the culture device is in a chip format, e.g., the chamber 101 and fluidic channels 102, 103 are fabricated or molded from a fabricated master that is brought to bear upon a substrate material such as silicon, polymeric material or the like, and which substrate material comprises the chip, such that the device is formed either as a single device upon a single chip, or as a modular system with one or more discrete devices formed upon a single chip. Generally the chip format is provided in a small scale, usually not more than about 10 cm. on a side, or even not more than about 5 cm. on a side. It may even be only about 2 cm. on a side, or smaller. The chamber 101 and fluidic channels 102, 103 are correspondingly micro-scale in size.

The device will usually include a mechanism for obtaining signals from the cells, subcellular components, or cellular products and culture medium. The signals from the chamber 101 and channels 102, 103 can be monitored in real time. For example, biosensors can be integrated or external to the device, which permit real-time readout of the physiological status of the cells in the system. The present invention provides an ideal system for high-throughput screening to identify positive or negative response to a range of substances such as, for example, pharmaceutical compositions, vaccine preparations, cytotoxic chemicals, mutagens, cytokines, chemokines, growth factors, hormones, inhibitory compounds, chemotherapeutic agents, and a host of other compounds or factors. The substance to be tested could be either naturally occurring or it could be synthetic, and it could be organic or inorganic. For example, the activity of a cytotoxic compound can be measured by its ability to damage or kill cells in culture. This may readily be assessed by vital staining techniques. The effect of growth/regulatory factors may be assessed by analyzing the cellular content of the matrix, e.g., by total cell counts, and differential cell counts. This may be accomplished using standard cytological and/or histological techniques including the use of immunocytochemical techniques employing antibodies that define type-specific cellular antigens. The metabolic by-products of a specific compound can be assessed by analyzing the culture medium by mass spectrometry or high-pressure liquid chromatography (“HPLC”) methods.

In one embodiment, the present invention may provide a system for screening or measuring the effects of various environmental conditions or compounds on a biological system. For example, air or water conditions could be mimicked or varied in the device. The impact of different known or suspected toxic substances could be tested. The present invention further provides a system for screening consumer products, such as cosmetics, cleansers, or lotions. It also provides a system for determining the safety and/or efficacy of nutriceuticals, nutritional supplements, or food additives. The present invention could also be used as a miniature bioreactor or cellular production platform to produce cellular products in quantity.

The present invention provides a novel device, systems, and methods as set forth within this specification. In general, all technical and scientific terms used herein have the same meaning as commonly understood to one of ordinary skill in the art to which this invention belongs, unless clearly indicated otherwise. For clarification, listed below are definitions for certain terms used herein to describe the present invention. These definitions apply to the terms as they are used throughout this specification, unless otherwise clearly indicated.

As used herein the singular forms “a”, “and”, and “the” include plural referents unless the context clearly dictates otherwise. For example, “a compound” refers to one or more of such compounds, while “the cell” includes a particular cell as well as other family members and equivalents thereof as known to those skilled in the art.

Physiologically-Based Culture System

An in vitro cell culture system, wherein the cells or subcellular material (e.g., subcellular components or cellular products) are maintained under conditions providing physiological parameter values that model those found in vivo. A physiologic culture device comprises fluidic channels 102, 103 connecting at least one chamber 101, where the specific chamber 101 geometry is designed to provide parametric values of cellular interactions, liquid flow rate, liquid flow volume, liquid residence time, shear stress and/or other physiological parameters that correlate with the values of those parameters as found in vivo in the corresponding cell(s), tissue(s), or organ system(s) that the chamber(s) 101 of the physiological culture device simulates in vitro. In one embodiment, the device is seeded with cells of a type drawn from, corresponding directly to, or otherwise representing the cells, organ or tissue being modeled—e.g., liver cells in a liver-simulative culture chamber 101, and the like—to comprise the culture system.

Pharmacokinetic-Based Culture System

A physiologically-based culture system, wherein the cells or subcellular material are maintained under conditions providing pharmacokinetic parameter values that model those found in vivo. A pharmacokinetic culture device comprises fluidic channels 102, 103 connecting at least one chamber 101, where the specific chamber 101 geometry is designed to provide parametric values of cellular interactions, liquid flow rate, liquid flow volume, liquid residence time, and/or other pharmacokinetic parameters that correlate with the values of those parameters as found in vivo in the corresponding cell(s), tissue(s), or organ system(s) that the chamber(s) 101 of the pharmacokinetic culture device simulates in vitro. In one embodiment, the device is seeded with cells of a type drawn from, corresponding directly to, or otherwise representing the cells, organ or tissue being modeled—e.g., liver cells in a liver-simulative culture chamber 101, and the like—to comprise the culture system.

In one embodiment, the culture systems of the invention provide for at least one pharmacokinetic parameter to have a value that is comparable to values obtained for the cell, tissue, or organ system of interest in vivo; preferably at least two parameters may have comparable values, and the embodiment may provide for three or more comparable parameter values. Pharmacokinetic parameters of interest include, for example, interactions between cells, liquid residence time, compound residence time, liquid-to-cell volume ratios, circulatory transit time, circulatory flow distribution, relative organ or tissue size, metabolism by cells, and the like.

By comparable values, it is meant that the actual values produced by the embodiment do not deviate more than 25% from the theoretical values generated by the PBPK, pharmacokinetic/pharmacodynamic (“PK/PD”), drug clearance, or other form of mathematical model based on which the design of the physical features of and the rate of fluid flow through the device (collectively, the geometry of the device) are determined. Drug clearance models are mathematical models used to predict the length of time a drug remains in the body and/or the rate of elimination of a drug from the blood. A PK/PD model is a mathematical model used to predict the action of a drug in a living system based on pharmacokinetic information derived from in silico, in vitro or animal data. For example, the liquid residence time in the lung compartment for a rat, as calculated in a PBPK model, is 2 seconds, and the actual value measured in the lung cell culture chamber 101 of a rat-simulative pharmacokinetic-based culture system was 2.5+/−0.7 seconds. In another embodiment of the culture device, the pharmacokinetic values may deviate by no more than 50% from the theoretical values.

In another embodiment of the culture device, the pharmacokinetic values may deviate by no more than 100% from the theoretical values. In another embodiment of the device, the actual value(s) may differ exponentially from the theoretical value(s) by no more than two orders of magnitude, stated algebraically as:
T×10⁻²<A<T×10²
where T is the theoretical value and A is the actual value.

In another embodiment of the device, the actual value(s) may differ exponentially from the theoretical value(s) by no more than three orders of magnitude. In another embodiment of the device, the actual value(s) may differ exponentially from the theoretical value(s) by no more than four orders of magnitude. In yet another embodiment, while the maximum percentage or order of magnitude of deviation of actual from theoretical value(s) for one or more pharmacokinetic parameters is not pre-determined or specified, and may not be known, the embodiment is mass-produced in such a way as to cause the amount of deviation to be substantially constant as between any one manufactured specimen or copy of the embodiment and another specimen or copy of that embodiment, thereby promoting substantially similar comparability of actual to theoretical values in operations performed on different specimens or copies of the same embodiment.

The pharmacokinetic parameter value is obtained by using the equations of a PBPK or other mathematical model. Such equations have been described in the art, for example see Poulin and Theil (2000) J Pharm Sci. 89(1):16-35; Slob et al. (1997) Crit Rev Toxicol. 27(3):261-72; Haddad et al. (1996) Toxicol Lett. 85(2):113-26; Hoang (1995) Toxicol Lett. 79(1-3):99-106; Knaak et al. (1995) Toxicol Lett. 79(1-3):87-98; and Ball and Schwartz (1994) Comput Biol Med. 24(4):269-76, herein incorporated by reference. Pharmacokinetic parameters can also be obtained from the published literature, for example see Buckpitt et al., (1984) J. Pharmacol. Exp. Ther. 231:291-300; DelRaso (1993) Toxicol. Lett. 68:91-99; Haies et al., (1981) Am. Rev. Respir. Dis. 123:533-541.

Specific pharmacokinetic parameters of interest include interactions between cells, liquid residence time in a tissue or organ, interactions between cells, relative tissue or organ mass, liquid-to-cell volume ratio, circulatory transit time, compound residence time in a tissue or organ, circulatory flow distribution, metabolism by cells, etc. Physiologically relevant parameter values can be obtained empirically according to conventional methods, or can be obtained from values known in the art and publicly available. Pharmacokinetic parameter values of interest are obtained for an animal—usually a mammal, although other animal models can also find use, e.g., insects, fish, reptiles, or avians. Mammals include laboratory animals, e.g., mouse, rat, rabbit, or guinea pig; mammals of economic value, e.g., equine, ovine, caprine, bovine, canine, or feline; primates, including monkeys, apes, or humans; and the like. Different values may be obtained and used for animals of different ages, e.g., fetal, neonatal, infant, child, adult, or elderly; and for different physiological states, e.g., diseased, after contact with a pharmaceutically active agent, after infection, or under conditions of altered atmospheric pressure; and representing different phenotypic variations.

In one embodiment, information relevant to the pharmacokinetic parameter values, as well as mass balance equations applicable to various substances to be modeled in the system, is provided in a data processing component of the culture system, e.g., look-up tables in general purpose memory set aside for data storage, and the like. These equations comprise one or more physiologically-based pharmacokinetic (“PBPK”) models describing the dynamics of various biological or chemical substances within physiological systems; or in an alternative embodiment, these equations may comprise one or more mathematical models, of type(s) other than PBPK models, of the dynamics of such substances in such systems.

In Vitro Culture Device

The culture device of an embodiment of the invention provides a substrate for cells, subcellular material, subcellular components, or cellular products. Each device comprises at least one chamber 101 connected by or otherwise integrated with fluidic channels 102, 103. The chamber(s) 101 can be on a single substrate or device or on different substrates or devices. The device may contain a reservoir or compartment for the addition or withdrawal of culture media. The device may contain a cover to seal the chamber 101 and channels 102, 103 and may comprise at least one inlet 104 and one outlet 105 that allows for recirculation of the culture medium. In one embodiment, the device contains a mechanism to pump 108 the culture medium through the system. The culture medium is designed to maintain viability of the cultured cells, subcellular components, or cellular products. In one embodiment, the device contains a mechanism by which test compounds can be introduced into the system. These features may be integrated 1) into the single compartment containing the cultured cells, subcellular components, or cell products, or 2) embodied through one or more additional compartments that do not contain cultured cells, subcellular components, or cell products, or through other features of the design.

The device may include a mechanism for obtaining signals from the cells, subcellular components, or cellular products and culture medium. The signals from the chamber 101 and channels 102, 103 can be monitored in real time. For example, biosensors can be integrated or external to the device, which permit real-time readout of the physiological status of the cells in the system.

The culture device of the present invention may be provided in microsystem form as a chip 100, or substrate. In addition to enhancing the fluid dynamics of the device, such Microsystems save on space, particularly when used in highly parallel systems, and can be produced inexpensively. The culture device can be formed from a polymer such as but not limited to polystyrene, and may be disposed of after one use, eliminating the need for sterilization. As a result, the in vitro system can be produced inexpensively and widely used. In addition, the cells may be grown in a three-dimensional manner, e.g., to form a tube, which more closely replicates the in vivo environment.

To model the metabolic response of an animal for any particular agent, an embodiment of the present invention may comprise a bank of parallel or multiplex arrays comprising a plurality (e.g., at least two) of the culture systems, where each system can be identical, or can be varied with predetermined parameter values or input agents and concentrations. The array may comprise fewer than 10, about 10, or any larger number of systems including as many as 100 or more systems. Advantageously, the culture systems on microchips 100 can be housed within a single incubator so that all the cell culture systems are exposed to the same conditions during an assay. Alternatively, multiple chips 100 may be interconnected to form a single device, e.g. to mimic gastrointestinal barriers or the blood-brain barrier.

Cells

Cells for use in the assays performed on the invention can be an organism, a multiplicity of cells of a single type derived from an organism, or they can be comprised of a mixture of cell types, as is typical of in vivo situations. The culture conditions may include predetermined values or value ranges of, for example, temperature, pH, presence of factors, presence of other cell types, and the like. A variety of animal cells can be used, including any of the animals for which pharmacokinetic parameter values can be obtained, as previously described.

The invention is suitable for use with any cell type, including primary cells, and both normal and transformed cell lines. The present invention is suitable for use with single cell types or cell lines; or combinations of different cell types thereof. Preferably the cultured cells maintain the ability to respond to stimuli that elicit a response in their naturally occurring counterparts. Cells used with the present invention may be derived from all sources such as eukaryotic or prokaryotic cells. The eukaryotic cells can be plant-derived in nature or animal-derived in nature, such as cells derived from humans, simians, or rodents. They may be of any tissue type (e.g., heart, stomach, kidney, intestine, lung, liver, fat, bone, cartilage, skeletal muscle, smooth muscle, cardiac muscle, bone marrow, muscle, brain, pancreas, cornea), and of any cell type (e.g., epithelial, endothelial, mesenchymal, adipocyte, hematopoietic). Further, a cross-section of tissue or an organ can be used. For example, a cross-section of an artery, vein, gastrointestinal tract, esophagus, or colon could be used. Further, cells or subcellular material that comprise an artificial tissue construct can be used.

In addition, cells that have been genetically altered or modified so as to contain a non-native “recombinant” nucleic acid sequence, or modified by antisense technology to provide a gain or loss of genetic function, may be utilized with the invention. Methods for generating genetically modified cells are known in the art, see for example “Current Protocols in Molecular Biology”, Ausubel et al., eds, John Wiley & Sons, New York, N.Y., 2000. The cells could be terminally differentiated or undifferentiated. The cells of the present invention could be cultured cells derived from a variety of genetically diverse individuals that may respond differently to biologic and pharmacologic agents. Genetic diversity can have indirect and direct effects on disease susceptibility. In a direct case, even a single nucleotide change, resulting in a single nucleotide polymorphism (SNP), can alter the amino acid sequence of a protein and directly contribute to disease or disease susceptibility. For example, certain APO-lipoprotein E genotypes have been associated with onset and progression of Alzheimer's disease in some individuals.

When certain polymorphisms are associated with a particular disease phenotype, cells from individuals identified as carriers of the polymorphism can be studied for developmental anomalies, using cells from non-carriers as a control. The present invention provides an experimental system for studying developmental anomalies associated with particular genetic disease presentations since several different cell types can be studied simultaneously, and linked to related cells. For example, neuronal precursors, glial cells, or other cells of neural origin, can be used in a device to characterize the cellular effects of a compound on the nervous system. Also, cell culture systems can be configured so that cells can be studied to identify genetic elements that affect drug sensitivity, chemokine and cytokine response, response to growth factors, hormones, and inhibitors, as well as responses to changes in receptor expression and/or function. This information can be invaluable in designing treatment methodologies for diseases of genetic origin or for which there is a genetic predisposition.

In one embodiment of the invention, the cells used in the in vitro culture device are cells involved in the detoxification and metabolism of pharmaceutically active compounds, e.g. liver cells, including hepatocytes.

The growth characteristics of tumors, and the response of surrounding tissues and the immune system to tumor growth are also of interest. Cells associated with degenerative diseases, including cells of both affected tissues and of surrounding areas, may be exploited in the system of the present invention to determine both the response of the affected tissue, and the interactions with other parts of the body.

The term “environment”, or “culture condition,” encompasses cells, media, factors, time and temperature. Environments may also comprise drugs and other compounds, particular atmospheric conditions, pH, salt composition, minerals, etc. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such a fetal calf serum, or media which is fully defined and serum-free. A variety of culture media are known in the art and commercially available.

Screening Assays

Drugs, toxins, cells, pathogens, samples, antigens, antibodies, etc., including engineered or synthetically created as well as naturally derived substances, herein referred to generically as “input variables,” are screened for biological activity by adding them to the pharmacokinetic-based culture system, and then assessing the cultured cells, subcellular components, or cellular products for changes in output variables of interest, e.g., consumption of O₂, production of CO₂, cell viability, expression of proteins of interest, activity of enzymes of interest, and the like. The input variables are typically added in solution, or readily soluble form, to the medium of cells in culture. The input variables may be added using a flow-through system, or alternatively, adding a bolus to an otherwise static solution. In a flow-through system, two fluids are used, where one is physiologically neutral solution, and the other is the same solution with the test compound added. The first fluid is passed over the cells, subcellular components, or cellular products, followed by the second fluid. In a single solution method, a bolus of the test input variables is added to the volume of medium surrounding the cells, subcellular components, or cellular products. The overall composition of the culture medium should not change significantly with the addition of the bolus, or between the two solutions in a flow-through method.

Preferred input variable formulations do not include additional components, such as preservatives, that have a significant effect on the overall formulation. Thus preferred formulations consist essentially of a biologically active agent and a physiologically acceptable carrier, e.g. water, ethanol, or DMSO. However, if an agent is liquid without an excipient the formulation may consist essentially of the compound itself.

A plurality of assays may be run in parallel with different input variable concentrations to obtain a differential response to the various concentrations. As known in the art, the process of determining the effective concentration of an agent typically uses a range of concentrations resulting from 1:10, or other log scale, dilutions. The concentrations may be further refined with a second series of dilutions, if necessary. Typically, one of these concentrations serves as a negative control, e.g. at zero concentration or below the level of detection.

Input variables of interest encompass numerous chemical classes, though frequently they are organic molecules. A preferred embodiment is the use of the methods of the invention to screen candidate agent samples, e.g. environmental samples or samples of pharmaceutical molecular entities, for toxicity. Candidate agents may comprise functional groups necessary for structural interaction, particularly hydrogen bonding, with proteins, and typically include at least one amine, carbonyl, hydroxyl or carboxyl group, and preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Input variables may also be inorganic molecules such as, for example, molecules that comprise industrial chemicals or consumer products like cosmetics.

Included among input variables of interest are pharmacologically active compounds or drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in The Pharmacological Basis of Therapeutics, Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds used as input variables include all of the classes of molecules described above, and may further comprise samples of unknown content. While many samples will comprise compounds in solution, solid samples that can be dissolved in a suitable solvent may also be assayed. Samples of interest include environmental samples, e.g., ground water, sea water, or mining waste; biological samples, e.g., lysates prepared from crops or tissue samples; manufacturing samples, e.g., time-course samples isolated during preparation of pharmaceuticals; as well as libraries of compounds prepared for analysis; and the like. Samples of interest include both synthetic and naturally occurring compounds being assessed for potential therapeutic value, e.g., drug candidates derived from plant or fungal cells, etc.

The term “samples” also includes the fluids described above to which additional components have been added, for example, components that affect the ionic strength, pH, or total protein concentration. In addition, the samples may be treated to achieve at least partial fractionation or concentration. Biological samples may be stored if care is taken to reduce degradation of the compound, e.g., under nitrogen, frozen, or a combination thereof. The volume of sample used is sufficient to allow for measurable detection; usually from about 0.01 ml. to 1 ml. of a biological sample is sufficient, although greater or lesser quantities may in some circumstances be employed.

Compounds and candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Output Variables

Output variables are quantifiable elements of cells, subcellular material, subcellular components, or cellular products, particularly elements that can be accurately measured in a high throughput assay system. An output can be a feature, condition, state or function of any cell, cellular component or cellular product including viability, respiration, metabolism, cell surface determinant, receptor, protein or conformational or posttranslational modification thereof, lipid, carbohydrate, organic or inorganic molecule, mRNA, DNA, etc., or a portion derived from such a cell component. While most outputs will provide a quantitative readout, in some instances a semi-quantitative or qualitative result will be obtained. Readouts may include a single determined value, or may include mean, median value or the variance, etc. Characteristically a range of readout values will be obtained for each output. Variability is expected and a range of values for a set of test outputs can be established using standard statistical methods.

Various methods can be utilized for quantifying the presence of selected markers of physiological conditions, processes or outcomes. For measuring the amount of a molecule that is present, a convenient method is to label the molecule with a detectable moiety, which may be fluorescent, luminescent, radioactive, enzymatically active, etc. Fluorescent and luminescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81).

Output variables may be measured by immunoassay techniques such as immunohistochemistry, radioimmunoassay (RIA), or enzyme linked immunosorbance assay (ELISA) and related non-enzymatic techniques. These techniques utilize specific antibodies as reporter molecules which are particularly useful due to their high degree of specificity for attaching to a single molecular target. Cell-based ELISA or related non-enzymatic or fluorescence-based methods enable measurement of cell surface parameters. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Data Analysis

The results of screening assays may be compared to results obtained from reference compounds, concentration curves, controls, etc. The comparison of results is accomplished by the use of suitable deduction protocols, artificial intelligence (“AI”) systems, statistical comparisons, etc.

One or more databases of reference output data can be compiled. These databases may include results from known agents or combinations of agents, as well as references from the analysis of cells treated under environmental conditions in which single or multiple environmental conditions or parameters are removed or specifically altered. A data matrix may be generated, where each point of the data matrix corresponds to a readout from a output variable, where data for each output may come from replicate determinations, e.g., multiple individual cells of the same type.

The readout may be a mean, average, median or the variance or other statistically or mathematically derived value associated with the measurement. The output readout information may be further refined by direct comparison with the corresponding reference readout. The absolute values obtained for each output under identical conditions will display a variability that is inherent in live biological systems and that may also reflect individual cellular variability as well as the variability inherent between individuals.

Cell Cultures and Cell Culture Devices

In one embodiment of the present invention, the culture devices of the invention comprise channels 102, 103, connecting to or otherwise integrated with at least one chamber 101, preferably integrated into a chip format. In one embodiment, the specific chamber geometry is designed, based on in vivo characteristics characterized by one or more parameters of a PBPK or other type of mathematical model, to provide cellular interactions, liquid flow, and liquid residence parameter values that correlate with the parameter values found in vivo for the corresponding cells, tissue, or organ systems being simulated. In another embodiment, the specific chamber geometry is not based on in vivo characteristics modeled by parameters of a PBPK or other type of mathematical model.

Optimized chamber geometries can be developed by reiterating the procedure of testing parameter values in response to changes in fluid flows and in physical features, arrangements and dimensions, until the desired values are obtained. One method of optimization of the culture device (e.g., the substrate) includes selecting the number of chambers 101, choosing a chamber geometry that provides the proper cell-to-volume ratio, choosing the particular internal topographical features of the chamber 101 or, if there be more than one, of each chamber 101, selecting a chamber size (or, if there be more than one chamber, the respective chamber sizes) that provides the proper relative tissue or organ size, choosing the optimal fluid flow rates that provide for the correct liquid residence time, then calculating the cell shear stress based on these values. If the cell shear stress is greater than the maximum allowable value, new parameter values are selected and the process is repeated.

Microprocessors can serve to compute a physiologically-based pharmacokinetic (PBPK) or other mathematical model for the kinetics or dynamics of a particular test chemical in a system. These calculations may serve as the basis for setting the flow rates among compartments and the excretion rates for the test chemical from the system comprised by the culture device. However, they may also serve as a theoretical estimate for the test chemical itself. At the conclusion of the experiment, predictions concerning the concentrations of test chemicals and metabolites made by the PBPK or other mathematical determination can be compared to the sensor data. Hard copy output generated by the device permits comparison of output from the PBPK or other mathematical model with experimental results.

Fabrication

The in vitro culture device typically comprises an aggregation of separate elements, e.g., chamber 101, channels 102, 103, inlet 104, or outlets 105, which when appropriately mated, joined, or otherwise integrated together, form the culture device of the invention. Preferably the elements are provided in an integrated, “chip-based” format.

The fluidics of a device are appropriately scaled for the size of the device. In a chip-based format, the fluidic connections may be “microfluidic”, e.g., a fluidic element, such as a passage, chamber 101 or conduit that has at least one internal cross-sectional dimension, e.g., depth or width, of less than 1 mm. In one embodiment of the present invention, the channels 102, 103 connecting the chamber 101 of the culture device typically include at least one microfluidic channel. In another embodiment of the present invention, none of the features of the device contain microfluidic channels.

Typically, culture devices comprise a top portion, a bottom portion, and an interior portion, wherein the interior portion substantially defines the channels 102, 103 and chamber 101 of the device. In preferred aspects, the bottom portion will comprise a solid substrate that is substantially planar in structure, and which has at least one substantially flat upper surface. A variety of substrate materials may be employed as the bottom portion. Because the devices can be microfabricated, substrate materials might be selected based upon their compatibility with known microfabrication techniques, e.g., photolithography, thin-film deposition, wet chemical etching, reactive ion etching, inductively coupled plasma deep silicon etching, laser ablation, air abrasion techniques, injection molding, embossing, and other techniques.

In additional preferred aspects, the substrate materials will comprise polymeric materials, e.g., plastics, such as polystyrene, polymethylmethacrylate (PMMA), polycarbonate, polytetrafluoroethylene (TEFLON®), polyvinylchloride (PVC), polydimethylsiloxane (PDMS), polysulfone, and the like. Such substrates are readily manufactured from masters, using well-known molding techniques, such as injection molding, embossing or stamping, or by polymerizing the polymeric precursor material within the mold. Such polymeric substrate materials are preferred for their ease of manufacture, low cost and disposability, as well as their general inertness to most extreme reaction conditions. Again, these polymeric materials may include treated surfaces, e.g., derivatized or coated surfaces, to enhance their utility in the system, e.g., provide enhanced fluid direction, cellular attachment or cellular segregation.

In one embodiment of the present invention, the channels 102, 103 and/or chamber(s) 101 of a culture device are typically fabricated into the upper surface of the substrate, or bottom portion, using the above described techniques, as grooves or indentations. The lower surface of the top portion of a culture device, which top portion typically comprises a second planar substrate, is then overlaid upon and bonded to the surface of the bottom substrate, sealing the channels 102, 103 and/or chamber(s) 101 (the interior portion) of the device at the interface of these two components. Bonding of the top portion to the bottom portion may be carried out using a variety of known methods, depending upon the nature of the substrate material. For example, in the case of glass substrates, thermal bonding techniques may be used which employ elevated temperatures and pressure to bond the top portion of the device to the bottom portion. Polymeric substrates may be bonded using similar techniques, except that the temperatures used are generally lower to prevent excessive melting of the substrate material. Alternative methods may also be used to bond polymeric parts of the device together, including acoustic welding techniques, or the use of adhesives, e.g., UV curable adhesives, and the like.

In one embodiment of the present invention, the device will generally comprise a pump 108, such as an electrokinetic pump. The pump 108 generally operates at flow rates on the order of 0.1 μL/min. The pump system can be any fluid pump device, such as a peristaltic pump or a diaphragm pump, etc. and can be either integral to the culture device (e.g., when the device comprises a chip-based system) or a separate component as described above. In one embodiment of the present invention, the device comprises more than one pump 108.

The device can be connected to or interfaced with a processor, which stores and/or analyzes the signal(s) from each the biosensors. The processor in turn forwards the data to computer memory (e.g., either hard disk or RAM) from where it can be used by a software program to further analyze, print and/or display the results. The computer memory may be local to the processor, or it may be situated elsewhere on a network including on the Internet.

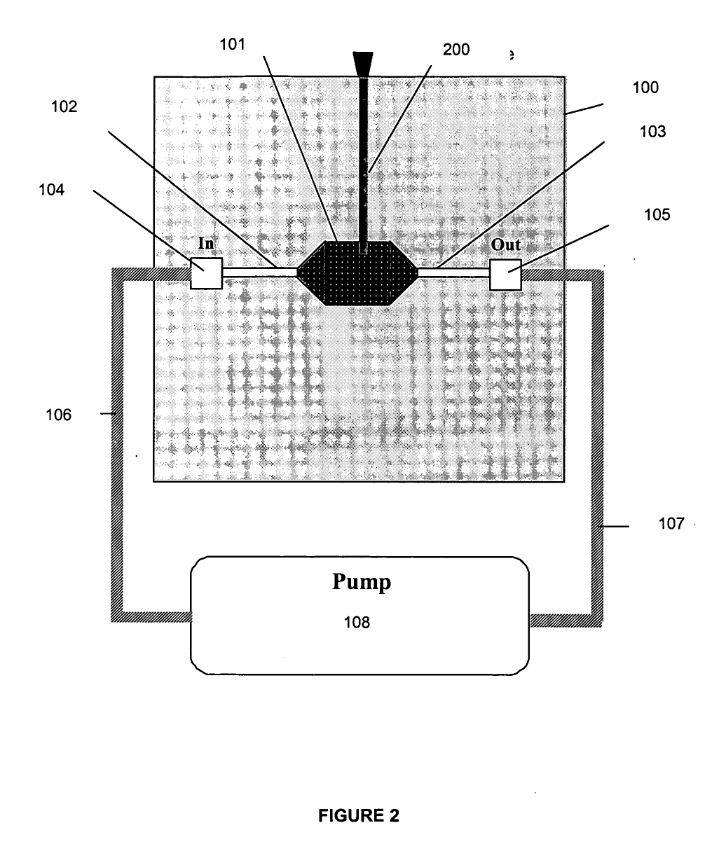

FIG. 2 is a schematic of another embodiment of the invention. In FIG. 2 a signal path is provided on the chip 100. Signals for monitoring various aspects of system can be taken from the chip 100 and at specific locations on the chip and moved to outputs off the chip. In one example, the signal path on the chip is an integrated buried waveguide 200. The chip, in such an embodiment, could be made of silicon, glass or a polymer. The waveguide carries light to the edge of the chip where a transducer is located to transform the light signal to an electrical signal. The cells, subcellular components, or cell products within the system can then be monitored for fluorescence, luminescence, or absorption or all these properties to interrogate and monitor the cells, subcellular components, or cell products within the system. Checking fluorescence requires a light source. The light source is used to interrogate the molecule and the signal carrier, such as a waveguide or a fiber optic captures the signal and sends it off the chip. The signal carrier would direct light to a photodetector near the end of the signal-carrying portion of the chip.

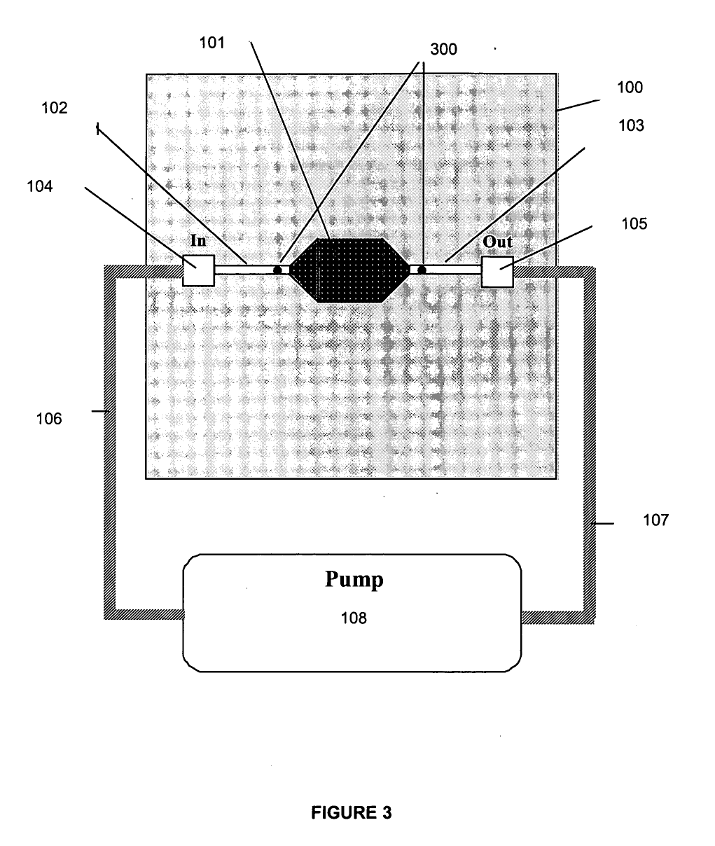

FIG. 3 is a schematic view of another embodiment of the system of the present invention. In this embodiment, biosensors 300 are positioned on the chip 100 upstream and downstream of the chamber 101 of the chip. The biosensors monitor the oxygen, carbon dioxide, and/or pH of the medium. These sensors allow monitoring of the system and adjustment of gas levels as needed to maintain a healthy environment. In addition, if positioned just upstream and downstream of each cell compartment, biosensors provide useful information on cellular metabolism, viability and/or enzyme activity.

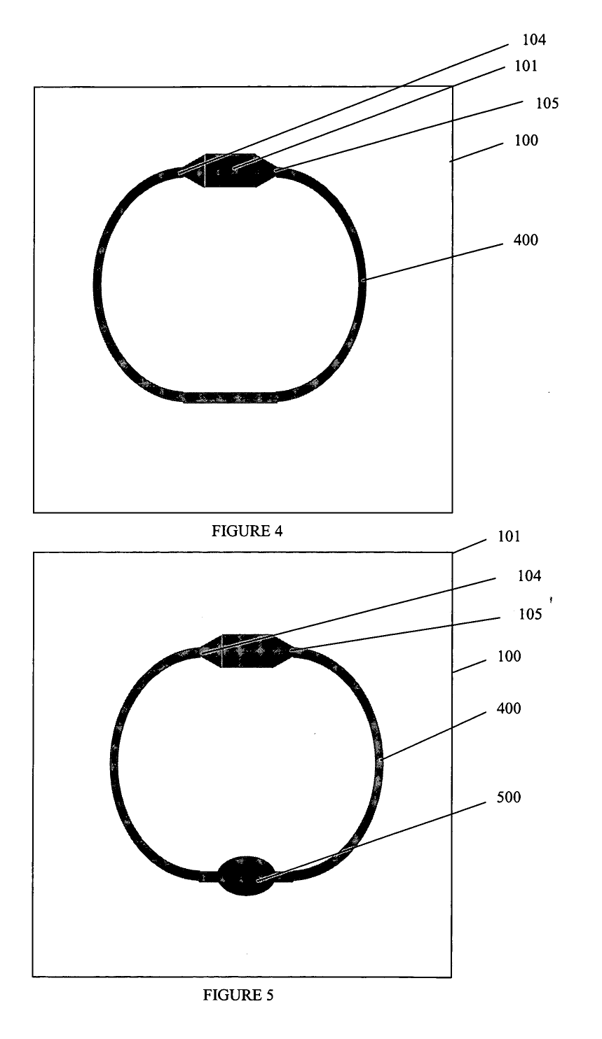

FIG. 4. is a schematic view of yet another embodiment of the system of the present invention. The system includes a culture chamber 101 formed on a substrate of silicon, which is commonly referred to as a chip 100. The chamber 101 has an inlet 104 and an outlet 105. The inlet 104 is located at one end of the chamber 101 and the outlet 105 is located at the other end of the chamber 101. The outlet 105 is connected to the inlet 104 by a fluid path, thus making a contiguous channel 400.

FIG. 5. is a schematic view of another embodiment of the system of the present invention. The system includes a culture chamber 101 containing an inlet 104 and an outlet 105 and a reservoir chamber 500 containing an inlet 104 and an outlet 105. The outlet 105 of the reservoir chamber 500 is connected to the inlet 104 of the culture chamber 101 by a fluid path 400 and the outlet 105 of the culture chamber 101 is connected to the inlet 104 of the reservoir chamber 500 by another fluid path 400.

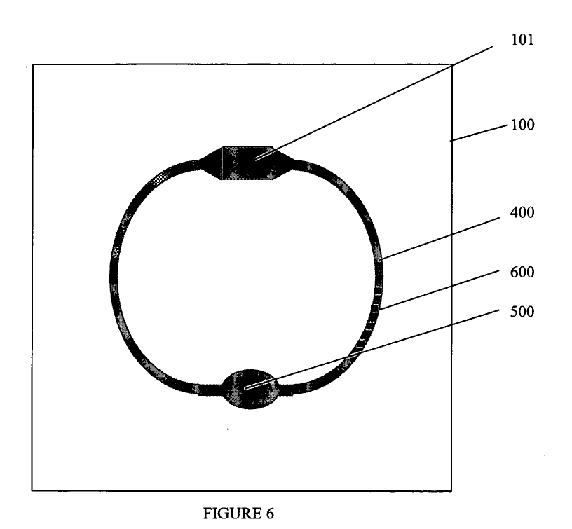

FIG. 6. is a schematic view of another embodiment of the system of the present invention. In this embodiment, the fluid channel 400 of the system contains electrodes (600) such that when a voltage is applied across two of these electrodes, fluid flows due to electrokinetic or electroosmotic flow. Voltage can be applied and alternated in sequence across the series of electrodes to induce directional flow of the culture medium.

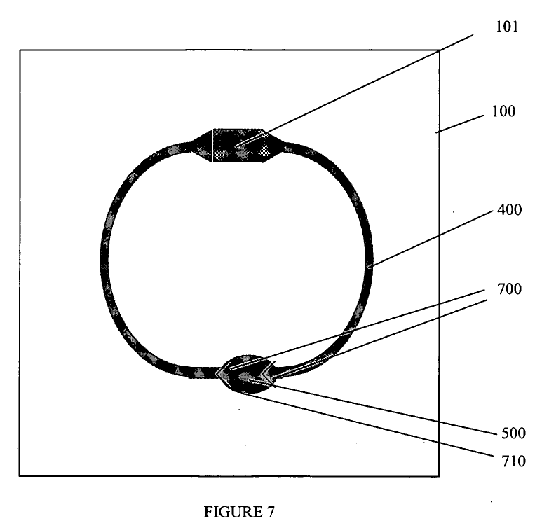

FIG. 7. is a schematic view of yet another embodiment of the system of the present invention. In this embodiment, the reservoir chamber (500) contains a one-way check valve (700) placed at the outlet 105 and another one-way check valve (700) placed at the inlet 104. A flexible silicone membrane (701) is placed over the reservoir chamber (500) and the culture chamber (101). The silicone membrane (701) over the reservoir chamber (500) is depressed downward, forcing fluid out of the reservoir chamber (500), through the fluid path (400), and into the culture chamber (101); when the silicone membrane over the reservoir is allowed to recover, fluid flows out of the culture chamber 101, through the fluid path, and into the reservoir chamber 500 through the inlet 104. This provides a diaphragm pumping mechanism that allows recirculating flow. It should be noted that there can be more than one reservoir chamber 500 or more than one culture chamber 101.

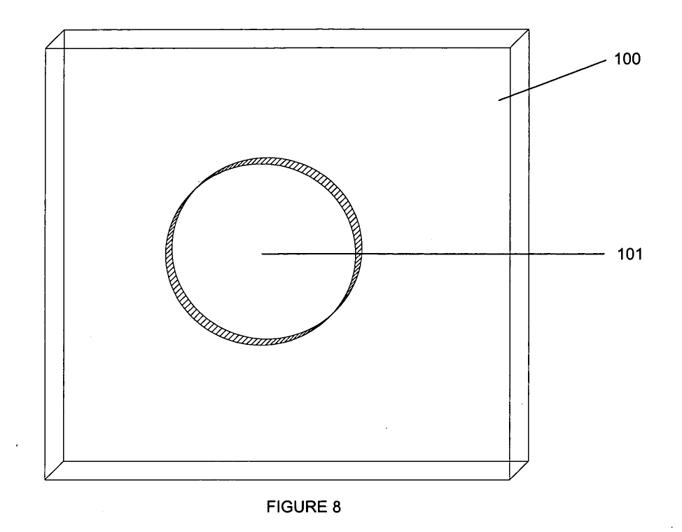

FIG. 8. is a schematic view of another embodiment of the system of the present invention. The system includes a culture chamber (101) formed on a substrate of silicon, which is commonly referred to as a chip (100). The chamber 101 contains cultured cells, cellular components, or cell products and an appropriate culture medium. Microscale magnetic particles with a density equal to or less than that of the culture medium are placed in the culture medium of the chamber 101. The chamber 101 is sealed with a gas permeable membrane. A circular flow is induced within the culture medium by placing the system in a rotating magnetic field.

### CONCLUSION

One embodiment of the present invention provides a pharmacokinetic-based culture device and system, usually including at least one chamber 101 having a receiving end and an exit end, and a conduit connecting the exit end to the receiving end. In one embodiment, the device is chip-based, e.g., it is microscale in size. A culture medium may be circulated through the culture chamber(s) 101 and through the conduit. The culture medium may also be oxygenated at one or more points in the recirculation loop.

The device may include a mechanism for communicating signals from portions of the device to a position off the chip, e.g., with a waveguide to communicate signals from portions of the device to a position off the chip.

The device for maintaining cells or subcellular material (e.g., subcellular components or cellular products) in a viable and/or functional state also includes a fluid circulation mechanism, which may be a flow-through fluid circulation mechanism or a fluid circulation mechanism which recirculates the fluid. The device for maintaining cells, subcellular components, or cellular products in a viable state also includes a fluid path. In one embodiment, a debubbler removes bubbles in the flow path. The device can further include a pumping mechanism. The pumping mechanism may be located on the substrate.

In one embodiment of the present invention, a method is provided for sizing a substrate to maintain cells or subcellular material (e.g., subcellular components or cellular products) in a viable and/or functional state in the chamber 101. The method includes the steps of determining the type of cells or subcellular material to be held on the substrate, and applying the constraints from a physiologically-based pharmacokinetic (“PBPK”) model to determine the physical characteristics of the substrate. The step of applying the constraints from a physiologically-based pharmacokinetic model includes determining the type of chamber 101 to be formed on the substrate, which may also include determining the geometry of the chamber 101 and determining the geometry of the flow path connecting to and from the chamber 101. The step of applying the constraints from a physiologically-based pharmacokinetic model may also include determining the composition of the fluid medium.

This embodiment of the present invention may be further specified by applying the constraints derived from the physiologically-based pharmacokinetic model to, alternatively, a single physiological parameter, to a plurality (e.g., more than one) of physiological parameters; or as further alternatives, by deliberately applying the constraints so that they do not produce parametric values that mimic or simulate the values of any corresponding parameter(s) as found in vivo, or by applying the constraints without regard to whether or not they produce parametric values that mimic or simulate the values of any corresponding parameter(s) as found in vivo.

All publications and patent applications cited in this specification are herein incorporated by reference as if each individual publication or patent application were specifically and individually indicated to be incorporated by reference.

It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A culture device comprising at least one microscale chamber that is configured to hold subcellular material, wherein the microscale chamber comprises an inlet and an outlet for fluid flow and wherein the microscale chamber is configured to simulate in vitro one or more physiological parameters derived from a mathematical model.

2. The culture device of claim 1, wherein, by virtue of its causing the simulation of at least one physiological parameter with a value comparable to a value obtained for that parameter in vivo, the geometry of the device tangibly embodies specific physiological information.

3. The culture device of claim 1, wherein the mathematical model is a physiologically-based pharmacokinetic model, or a single-compartment pharmacokinetic model, or a multi-compartment pharmacokinetic model, or a non-linear pharmacokinetic model, or a drug clearance model, or the like.

4. The culture device of claim 1, wherein the physiological parameter is a pharmacokinetic parameter.

5. The culture device of claim 4, wherein the geometry of the microscale chamber causes the device to simulate at least one pharmacokinetic parameter with a value comparable to a value obtained in vivo.

6. The culture device of claim 1, wherein the flow rate of fluid through the microscale chamber simulates at least one physiological parameter with a value comparable to a value obtained in vivo.

7. The culture device of claim 1, wherein the flow rate of fluid through the microscale chamber simulates at least one physiological parameter with a value less than or equal to a defined maximum value for that physiological parameter.

8. The culture device of claim 1, further comprising a second microscale chamber in fluidic communication with the first microscale chamber, wherein the second microscale chamber comprises an open reservoir for the addition or withdrawal of culture medium.

9. The culture device of claim 8 further comprising a third microscale chamber in fluidic communication with the first and second microscale chambers, wherein the third microscale chamber comprises a pumping mechanism.

10. The culture device of claim 1, further comprising culture medium.

11. The culture device of claim 10, wherein the culture medium flows through the microscale chamber.

12. The culture device of claim 10, wherein the culture medium re-circulates through the microscale chamber.

13. The culture device of claim 1, further comprising a pumping mechanism.

14. The culture device of claim 13, wherein the pumping mechanism is integrated in the culture device.

15. The culture device of claim 13, wherein the pumping mechanism is electrokinetic.

16. The culture device of claim 13, wherein the pumping mechanism is a diaphragm pump.

17. The culture device of claim 13, wherein the pumping mechanism is mechanically actuated.

18. The culture device of claim 13, wherein the pumping mechanism is pneumatically actuated.

19. The culture device of claim 13, wherein the pumping mechanism is external to the device.

20. The culture device of claim 1, further comprising a microfluidic channel in communication with the microscale chamber.

21. The culture system of claim 1, wherein the microscale chamber and the microfluidic channel are one and the same.

22. The culture device of claim 1, wherein the microfluidic channel comprises a debubbler located therein.

23. The culture device of claim 1, further comprising a debubbler that is located externally to the device.

24. The culture device of claim 4, wherein the pharmacokinetic parameter is selected from the group consisting of liquid residence time in a tissue or organ, compound residence time in a tissue or organ, interactions between cells, liquid to cell volume ratio, organ/tissue size ratio, circulatory transit time, circulatory flow distribution, and metabolism by cells.

25. The culture device of claim 1, further comprising at least one sensor for obtaining signals from the cellular medium.

26. The culture device of claim 25, wherein the at least one sensor is a biosensor.

27. The culture device of claim 25, wherein the at least one sensor comprises a waveguide.

28. The culture device of claim 1, wherein the device is microfabricated.

29. The culture device of claim 1, wherein the culture device is manufactured from a microfabricated master.

30. The culture device of claim 1, wherein the device is manufactured by mass production that causes the geometry of the device (including the provision for the rate of fluid flow in and through the device), and therefore the information embodied in the device, to be substantially the same from one such manufactured copy, specimen or iteration of the device to the next.

31. The culture device of claim 30, wherein the process of mass production includes that the device is manufactured from a microfabricated master.

32. The culture device of claim 1, wherein the microscale chamber provides for three-dimensional growth of cells.

33. The culture device of claim 1, wherein the microscale chamber contains a plurality of cell types.

34. The culture device of claim 1, wherein the microscale chamber contains a tissue biopsy.

35. The culture device of claim 1, wherein the microscale chamber contains a cross-section of a tissue or organ.

36. The culture device of claim 1, wherein the microscale chamber contains an artificial tissue construct.

37. The culture device of claim 1, wherein the microscale chamber comprises an artificial tissue construct.

38. The culture device of claim 1, wherein the subcellular material is a cellular product.

39. The culture device of claim 38, wherein the cellular product is selected from the group consisting of an enzyme, a nucleic acid, a protein, a lipid, and a carbohydrate.

40. The culture device of claim 38, wherein the cellular product is man-made.

41. The culture device of claim 38, wherein the cellular product comprises a naturally occurring or man-made cellular product in conjunction with some other biochemical entity.

42. The culture device of claim 1, wherein the subcellular material comprises a subcellular component.

43. The culture device of claim 42, wherein the subcellular component is a microsome, mitochondrion, nucleus, ribosome, plasma membrane, and the like.

44. The culture device of claim 42, wherein the subcellular component is man-made.

45. The culture device of claim 42, wherein the subcellular component comprises a naturally occurring or man-made subcellular component in conjunction with some other biochemical entity.

46. The culture system of claim 1, comprising multiple interconnected culture devices.

47. A method for culturing subcellular material comprising:
receiving subcellular material within a microscale chamber, wherein the microscale chamber comprises an inlet and an outlet for fluid flow through the microscale chamber; and
simulating in vitro one or more physiological parameters derived from a mathematical model.

48. The method of claim 47, wherein the mathematical model is a physiologically-based pharmacokinetic model.

49. The method of claim 47, wherein the physiological parameter is a pharmacokinetic parameter.

50. The method of claim 47, wherein the act of simulating simulates at least one pharmacokinetic parameter with a value comparable to a value obtained in vivo.

51. The method of claim 47, further comprising supplying the culture medium within the microscale chamber from a second microscale chamber in fluidic communication with the first microscale chamber, wherein the second microscale chamber comprises an open reservoir.

52. The method of claim 47, further comprising re-circulating a culture medium through the microscale chamber.

53. The method of claim 47, wherein the at least one pharmacokinetic parameter is selected from the group consisting of liquid residence time in a tissue or organ, compound residence time in a tissue or organ, interactions between cells, liquid to cell volume ratio, organ/tissue size ratio, circulatory transit time, circulatory flow distribution and metabolism by cells.

54. The method of claim 47 further comprising:
contacting the culture system with an input variable; and
monitoring at least one output parameter.

55. The method of claim 54, wherein the act of monitoring the output parameter comprises obtaining information from at least one sensor.

56. The method of claim 54, wherein the input variable is an organic compound.

57. The method of claim 54, wherein the input variable is an inorganic compound.

58. The method of claim 54, wherein the input variable is a complex sample.

59. The method of claim 54, wherein the input variable is selected from the group consisting of a pharmaceutical, environmental sample, a nutritional sample, or a consumer product, industrial chemical, biologically derived compound, biological and chemical warfare agent.

60. The method of claim 54, further comprising sensing the condition of the cellular medium.

61. A culture device comprising:
at least one microscale chamber that is configured to hold cellular material, wherein the microscale chamber comprises an inlet and an outlet for fluid flow and wherein the microscale chamber is configured to simulate in vitro one or more physiological parameters derived from a mathematical model;
a first sensor located upstream of the inlet of the microscale chamber;
a second sensor located downstream of the outlet of the microscale chamber; and
a culture medium that flows through the inlet and outlet of the microscale chamber.

62. The culture device of claim 61, wherein the first and second sensors are integrated buried waveguides.

63. The culture device of claim 61, wherein the at least one of the first and second sensors is a biosensor.

64. The culture device of claim 61, wherein the biosensor provides information on cellular metabolism.

65. The culture device of claim 61, wherein the biosensor provides information on enzyme activity.

66. The culture device of claim 61, wherein the first and second sensors are configured to monitor the culture medium.

67. The culture device of claim 66, wherein the first and second sensors are configured to monitor one of the group consisting of oxygen, carbon dioxide, and pH of the culture medium.

68. The culture device of claim 61, wherein the first and second sensors are configured to control gas levels within the microscale chamber.

69. A method for culturing cellular material comprising:
receiving cellular material in at least one microscale chamber, wherein the microscale chamber comprises an inlet and an outlet for fluid flow;
simulating in vitro one or more physiological parameters derived from a mathematical model;
sensing culture medium with a first sensor located upstream of the inlet of the microscale chamber; and
sensing the culture medium with a second sensor located downstream of the outlet of the microscale chamber.

70. The method of claim 69, wherein at least one of the acts of sensing obtains information on cellular metabolism.

71. The method of claim 69, wherein at least one of acts of sensing obtains information on enzyme activity.

72. The method of claim 69, wherein at least one of the acts of sensing monitors the culture medium.

73. The method of claim 69, wherein at least one of the acts of sensing monitors one of the group consisting of oxygen, carbon dioxide, and pH of the culture medium.

74. The method of claim 69, wherein at least one of the acts of sensing controls gas levels within the microscale chamber.

75. A culture device comprising:
at least one microscale chamber that is configured to hold cellular material, wherein the microscale chamber comprises an inlet and an outlet for fluid flow and wherein the microscale chamber is configured to simulate in vitro one or more physiological parameters derived from a mathematical model;
a fluid channel in fluidic communication with either the inlet or outlet of the microscale chamber; and
one or more electrodes in communication with the fluid channel, the one or more electrodes configured to induce fluid flow within the fluid channel.

76. The culture device of claim 75, further comprising a voltage source that is configured to alternate the sequence of voltage applied to the electrodes to induce directional flow of the fluid within the fluid channel.

77. The culture device of claim 75, wherein the electrodes induce eletrokinetic flow.

78. The culture device of claim 75, wherein the electrodes induce eletroosmotic flow.

79. A method for culturing cellular material comprising:
holding cellular material in at least one microscale chamber, wherein the microscale chamber comprises an inlet and an outlet for fluid flow;
simulating in vitro one or more physiological parameters derived from a mathematical model; and
altering voltage in one or more electrodes to induce flow fluid through the microscale chamber.

80. The method of claim 79, wherein the act of altering alternates the sequence of voltage applied to the electrodes to induce directional flow of the fluid within a fluid channel that is in fluidic communication with the microscale chamber.

81. The method of claim 79, wherein the act of altering voltage induces eletrokinetic flow.

82. The method of claim 79, wherein the act of altering voltages induces eletroosmotic flow.

83. A culture device comprising:
at least one microscale chamber that is configured to hold cellular material, wherein the microscale chamber comprises an inlet and an outlet for fluid flow, wherein the fluid flows through the microscale chamber, and wherein the microscale chamber is configured to simulate in vitro one or more physiological parameters derived from a mathematical model; and
at least one reservoir in fluidic communication with the microscale chamber, the reservoir comprising a flexible membrane, wherein depressing the flexible membrane induces fluid flow into the microscale chamber.

84. The culture device of claim 83, wherein the flexible membrane comprises silicon at least in part.

85. The culture device of claim 83, wherein the flexible membrane recirculates fluid flow between the microscale chamber and the reservoir.

86. The culture device of claim 83, wherein multiple reservoirs are in fluidic communication and at least one of the multiple reservoirs comprises the flexible membrane.

87. A method for culturing cellular material comprising:
holding cellular material within at least one microscale chamber wherein the microscale chamber comprises an inlet and an outlet for fluid flow, wherein the fluid flows through the microscale chamber;
simulating in vitro one or more physiological parameters derived from a mathematical model; and
inducing fluidic flow within the microscale chamber by depressing a flexible membrane.

88. The method of claim 87, wherein the flexible membrane is attached to a reservoir that is in fluidic communication with the microscale chamber.

89. The method of claim 87, wherein the flexible membrane comprises silicon at least in part.

90. The method of claim 87, wherein the act of inducing fluidic flow recirculates fluid flow between the microscale chamber and a reservoir.

91. A culture device comprising:
at least one microscale chamber that is configured to hold cellular material, wherein the microscale chamber comprises an inlet and an outlet for fluid flow and wherein the microscale chamber is configured to simulate in vitro one or more physiological parameters derived from a mathematical model; and
a culture medium within the microscale chamber, the culture medium comprising microscale magnetic particles.

92. The culture device of claim 91, further comprising a rotating magnetic field that induces a circular flow of the culture medium within the microscale chamber.

93. The culture device of claim 91, further comprising a magnetic field that induces a flow of the culture medium within the microscale chamber.

94. The culture device of claim 91, further comprising a gas permeable membrane that encloses at least a portion of the microscale chamber.

95. A method for culturing cellular material comprising:
holding cellular material in at least one microscale chamber, wherein the microscale chamber comprises an inlet and an outlet for fluid flow;
simulating in vitro one or more physiological parameters derived from a mathematical model; and
adding a culture medium to the microscale chamber wherein the culture medium comprises microscale magnetic particles.

96. The method of claim 95, further comprising rotating a magnetic field to induce a circular flow of the culture medium within the microscale chamber.

97. The method of claim 95, further comprising inducing a magnetic field that induces a flow of the culture medium within the microscale chamber.

98. The method of claim 95, further comprising enclosing at least a portion of the microscale chamber with a gas permeable membrane.
